(19) United States
(12) Patent Application Publication
(10) Pub. No.: US 2021/0346631 A1
(43) Pub. Date: Nov. 11, 2021

(54) PROTECTIVE HELMET FOR MEDICAL STAFF

(71) Applicant: Yizong He, Forest Lake, MN (US)

(72) Inventor: Yizong He, Forest Lake, MN (US)

(21) Appl. No.: 16/869,089

(22) Filed: May 7, 2020

(51) Int. Cl. A61M 16/06; A61M 16/00; A61M 16/20

(52) U.S. Cl. CPC A61M 16/0627; A61M 16/009; A61M 16/021; A61M 2209/088; A61M 2202/206; A61M 2205/8206; A61M 16/208

(57) ABSTRACT

A protective helmet, such as an antivirus helmet with a viewing window completely encloses the user's head. The entire helmet is airtight except for an air inlet and air outlet on its side wall. In order to prevent the viewing window from fogging by the user's exhaled gas, the window is made of sandwich glass with central heater wire to warm the glass. The air needed for the user to breathe is supplied by an external high pressure air reservoir. The air undergoes a pressure reduction and cooling process. Such an antivirus helmet provides safe and comfortable air for medical staff to breathe during their work in an infectious disease ward.

### BACKGROUND OF THE INVENTION

### Field of the Invention

This invention relates to medical apparatus and instruments, and in particular, it relates to a protective helmet system, such as an antivirus helmet system.

### Description of Related Art

There is no doubt that there are many ways infectious diseases can spread. Accordingly, various preventions should be taken to cope with a specific mode of transmission. For a pandemic, such as Covid-19, the infection form are still not exactly known. However, one known way that the coronavirus spreads is through normal breathing of infected humans. It has been proven that the coronavirus floats in air for quite a long time. As patients are treated in hospitals, it is inevitable that medical staff will be in close contact with patients. At the present, the common way to protect doctors and nurses from being infected is for them to wear face masks and safety goggles. In spite of this protective gear, a proportion of the medical staff may catch the virus. Traditional masks and goggles can only provide a limited degree of protection. There is no perfect way to completely avoid being infected with the virus. In addition, wearing a mask makes the user feel suffocated.

### SUMMARY

To address this problem, a protective helmet, such as an antivirus helmet including a helmet and a backpack is provided to protect medical staff from the virus spread by breathing the air in the place where they work. A healthy air supply, free from the virus, is stored in the air reservoir, and flows into a helmet for medical staff to breathe freely during their contact with patients. The antivirus helmet is a much safer protective device than face masks and goggles.

The antivirus helmet system according to embodiments of the present invention includes an airtight helmet, air and electricity supply system. The helmet encloses the user's head and is airtight except for an inlet and outlet. The helmet has an observation window made of sandwich glass or other transparent materials. Some heater wires are embedded in the sandwich glass; electricity is supplied by an external battery. The air that the user breathes is provided by an external air reservoir. The high pressure air free from virus is depressurized and cooled a bit before entering the helmet. To prevent the virus from entering the helmet against the exhaling current, the user's exhaled gas passes through a breathing valve and a water tank before being discharged into the ward. The air reservoir, battery, refrigerator and water tank are all placed in a backpack carried by the user. Such an antivirus helmet can provide a safe and comfortable breathing environment for medical staff working in a virus ward.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a protective helmet system which includes: a helmet, including a rigid enclosure with a transparent viewing window, and a retaining ring joined to the enclosure in an airtight connection, wherein the transparent viewing window includes multiple layers of transparent materials with embedded heater wire, and wherein the enclosure an air inlet hole and an air outlet hole in its side wall; and a backpack, wherein the backpack contains an air and electricity supply system coupled to the helmet.

In some embodiments, the rigid enclosure includes an upside-down cylindrical container and an upside-down frustum joined to the upside-down cylindrical container in an airtight connection, and wherein the retaining ring includes an elastic band.

In some embodiments, the air and electricity supply system includes: an air reservoir with an pressure reducing valve, configured to storing high pressure air; an air cooler; and a battery; wherein the air reservoir is connected to the air cooler, and the air cooler is connected to the air inlet hole of the helmet by a first tube, and wherein the battery is electrically coupled to the air cooler and the heater wire of the helmet.

It is to be understood that both the foregoing general description and the following detailed description are exemplary and explanatory and are intended to provide further explanation of the invention as claimed.

### BRIEF DESCRIPTION OF THE DRAWINGS

FIG. 1 schematically illustrates a helmet of an antivirus helmet system according to an embodiment of the present invention.

FIG. 2 shows apparatuses installed in the backpack for supplying air and power to the antivirus helmet according to an embodiment of the present invention.

### DETAILED DESCRIPTION OF PREFERRED EMBODIMENTS

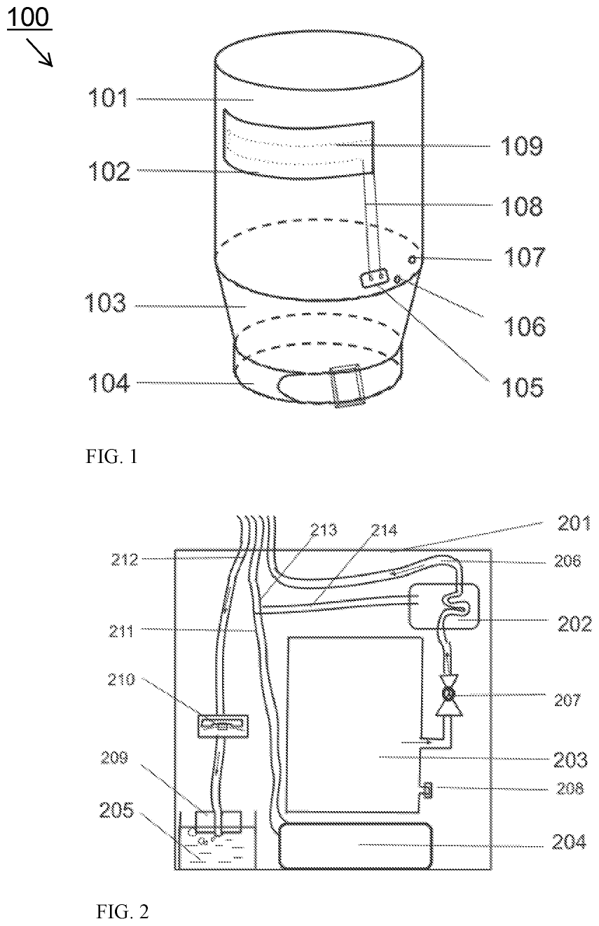

FIG. 1 schematically illustrates the helmet 100 of an antivirus helmet system. The light weight, thin material enclosure forms a cylindrical, inverted container 101, which is connected to an inverted frustum 103 below it in an airtight way, forming a rigid enclosure. Also, the inverted frustum 103 joins to a retaining ring 104 in an airtight way. The retaining ring 104 can be made of an elastic band which can be adjusted flexibly, according to the size of the user's neck. There is a viewing window 102 on the helmet corresponding to the eye position. Considering the possibility of fogging on the glass surface, the viewing window 102 is made of sandwich glass (i.e. multiple layers of glass). The heater wire 109 embedded in the sandwich glass is connected to the power socket 105, through the wire 108 in order to heat the glass. The window may also be made of other transparent materials. Two holes are drilled into the side wall of the helmet container 101: an air inlet 106 and an air outlet 107. They are linked to the backpack 201.

FIG. 2 shows the supply system of fresh air and electricity to the helmet 100. A high pressure air reservoir 203, battery 204, miniature refrigerator 202 and small water tank 205 are all contained in a backpack 201 carried by the user. Virus free air is pressed into the air reservoir 203 through the inlet valve 208. The high pressure air flows through the pressure reducing valve 207, then into the miniature refrigerator 202, and finally through the soft tube 206 into the helmet for the user to breathe. The battery 204 powers the miniature refrigerator 202 and the heater wires 109 through wire 214 and 213, respectively, and wire 211. The air coming out from the helmet 100 enters the outlet tube 212 through a breathing valve 210, a small water tank 205 and ends up in the air outside of the unit. The lower end of the tube 212 is kept below the water surface by a retainer block 209. The breathing valve 210 and the water in the water tank 205 are used to prevent the air in the ward from pouring into the helmet 100. The user may adjust the pressure reducing valve 207 and the miniature refrigerator 202 switch to optimize both the flow and temperature of the air before it enters into the helmet 100. Adjusting the battery 204 switch makes the window's heating system suitable to prevent the viewing window 102 from fogging. After these operations are completed, the user (e.g. medical staff) will get a safe and comfortable breathing environment because the user is not breathing the air of their working room, but air from the air reservoir 203. This will completely eliminate the possibility of getting infected with the virus through breathing.

It will be apparent to those skilled in the art that various modifications and variations can be made in the helmet system of the present invention without departing from the spirit or scope of the invention. Thus, it is intended that the present invention cover modifications and variations that come within the scope of the appended claims and their equivalents.

What is claimed is:

1. A protective helmet system comprising:
a helmet, including a rigid enclosure with a transparent viewing window, and a retaining ring joined to the enclosure in an airtight connection, wherein the transparent viewing window includes multiple layers of transparent materials with embedded heater wire, and wherein the enclosure an air inlet hole and an air outlet hole in its side wall; and
a backpack, wherein the backpack contains an air and electricity supply system coupled to the helmet.

2. The protective helmet system of claim 1, wherein the rigid enclosure includes an upside-down cylindrical container and an upside-down frustum joined to the upside-down cylindrical container in an airtight connection, and wherein the retaining ring includes an elastic band.

3. The protective helmet system of claim 1, wherein the air and electricity supply system includes:
an air reservoir with an pressure reducing valve, configured to storing high pressure air;
an air cooler; and
a battery;
wherein the air reservoir is connected to the air cooler, and the air cooler is connected to the air inlet hole of the helmet by a first tube, and
wherein the battery is electrically coupled to the air cooler and the heater wire of the helmet.

4. The protective helmet system of claim 3, wherein the air and electricity supply system further includes a water tank, wherein the water tank is connected to the air outlet of the helmet by a second tube, and wherein the second tube is equipped with a breathing valve.
